SEQUENCE LISTING (7 sequences)

SEQ ID NO: 1 - protein, 5 aa, Artificial Sequence
  synthetic peptide
RPPRS

SEQ ID NO: 2 - protein, 4 aa, Artificial Sequence
  synthetic peptide
RPPR

SEQ ID NO: 3 - protein, 4 aa, Artificial Sequence
  synthetic peptide
KPPK

SEQ ID NO: 4 - protein, 4 aa, Artificial Sequence
  synthetic peptide
KPGR

SEQ ID NO: 5 - protein, 4 aa, Artificial
  synthetic peptide
KGPR

SEQ ID NO: 6 - protein, 4 aa, Artificial Sequence
  synthetic peptide
RGPR

SEQ ID NO: 7 - protein, 4 aa, Artificial Sequence
  synthetic peptide
RGGR

(19) United States
(12) Patent Application Publication
(10) Pub. No.: US 2010/0113368 A1
(43) Pub. Date: May 6, 2010

(54) CHOLESTEROL LOWERING PROTEIN HYDROLYSATES

(76) Inventor: Luppo Edens, Rotterdam (NL)

Correspondence Address: NIXON & VANDERHYE, PC, 901 NORTH GLEBE ROAD, 11TH FLOOR, ARLINGTON, VA, 22203, US

(21) Appl. No.: 12/532,193

(22) PCT Filed: Mar. 18, 2008
(86) PCT No.: PCT/EP2008/053197

§ 371 Date: Sep. 21, 2009

(30) Foreign Application Priority Data
Mar. 21, 2007 (EP) 07104562.9

(51) Int. Cl. A61K 38/08; C07K 5/10; C07K 7/06; C12P 21/00; A61K 38/07; A61P 9/00

(52) U.S. Cl. 514/16; 530/330; 530/329; 530/328; 435/68.1; 514/18; 514/17

(57) ABSTRACT

The present invention relates to the production of novel peptides which have a length of between 4 and 8 amino acids and comprises the amino acid sequence -Xaa-Xbb-Xcc-Xdd-, whereby Xaa and Xdd can be His (H), Arg (R) or Lys (K), and Xbb and Xcc can be Pro (P) or Gly (G).

### FIELD OF THE INVENTION

The present invention relates to the production of novel peptides.

### BACKGROUND OF THE INVENTION

In the Western world, more than 95% of the dietary fat is long-chain triglycerides, with the remaining part largely made up of phospholipids, fatty acids and cholesterol. Because elevated plasma cholesterol levels (hypercholesterolemia or hyperlipidemia) are widely regarded as a primary risk factor for artherosclerosis and coronary heart disease, lowering of cholesterol intake is of broad medical importance. Plasma cholesterol levels are determined by the balance between diet, cholesterol biosynthesis, cholesterol secretion in bile acids from the liver, and cholesterol absorption and reuptake by the small intestine.

The overall whole-body cholesterol balance is kept mainly by matching fecal losses with endogenous cholesterol synthesis. Because of its very low water solubility, cholesterol uptake is completely dependent upon special transport mechanisms. The successive steps in the process of intestinal cholesterol digestion and absorption have been reviewed by Ros (Artherosclerosis 2000, 151, 357-379). Briefly, the peristaltic action of the stomach leads to an initial emulsion of water-soluble and non-water soluble dietary compounds. This emulsion is stabilized by a.o. dietary phospholipids and free fatty acids and monoglycerides formed by the gastric lipase. Once in the duodenum, the stomach contents interacts with specific receptors to stimulate the secretion of bile and pancreatic juice. Major components of bile are bile acids, phospholipids, cholesterol, proteins and bile pigments (Tuchweber et al., Nutr. Res. 1996, 16, 1041-1080). The pancreatic juice supplies the lipase that leads to a further breakdown of dietary fats as well as proteases leading to a breakdown of the dietary proteins into peptides and free amino acids. Stimulated by an increasing pH value due to bicarbonate secretion and the increasing concentrations of bile, lipophilic products present in the stomach content become increasingly solubilized in the form of micelles. The formation of these bile acid micelles are the first steps in a complex process that eventually results in the transfer of cholesterol and the hydrolysed dietary fats over the intestinal wall to end up in the blood.

A relatively large amount of cholesterol in the body is of dietary origin and taken up from the gut in the form of bile salt micelles or small unilamellar phospholipid vesicles according to the above described mechanism. By interfering the process of micellar cholesterol solubilization, the uptake of dietary cholesterol can be limited and hereby plasma cholesterol levels. WO 01/24789 describes socalled beta-peptides that are suitable as active substances that reduce cholesterol- and lipid-uptake from the gut. Beta-peptides consist of beta-amino acids which have an amino group in the beta-position. Beta-peptides form a more stable helix than natural (alpha) peptides do, but, more importantly, beta-peptides are stable against proteolytic degradation. According to WO 01/24789 the beta-peptides should be at least about 6 residues long and have at least two positive charges or should be amphipatic. The mechanism provided is that these beta-peptides act as a precipitant for cholesterol and lipids comprising micelles and vesicles having a negative surface charge. Their data show that polylysine as well as beta-peptides lead to an effective cholesterol precipitation whereas free lysine and free arginine, carrying only a single positive charge, do not.

Apart from such beta-peptides, also intact dietary proteins such as soybean protein and their hydrolysates are known to influence serum cholesterol levels. Cholesterol lowering effects have been described for soybean, casein and whey hydrolysates. From the whey protein beta-lactoglobulin specific hypocholesterolemic peptides have been described (Nagaoka et al., Biochem. Biophys. Res. Commun. 2001, 281, 11-17). Among the four peptides able to inhibit cholesterol absorption in vitro, peptide Ile-Ile-Ala-Glu-Lys could be shown also to be active in vivo. The latter peptide was selected for the in vivo test because only this peptide has no peptic or tryptic digestive site. Another publication (Yoshikawa et al., BioFactors 2000, 12, 143-146) mentions alpha-lactotensin, the beta-lactoglobulin derived peptide His-Ile-Arg-Leu, as a peptide capable of reducing serum cholesterol. The same publication refers to Leu-Pro-Tyr-Pro-Arg as a serum cholesterol reducing peptide obtained from soybean glycinin. In another study (Dae Young Kwon et al., Food Sci Biotechnol 2002, 11 (1) 55-61)) the hypocholesterolemic quality of the latter peptide was confirmed. According to these and other data, hydrophobicity is a factor of prime importance for bile binding and thus cholesterol lowering activity of these cholesterol loweing peptides. The presence of the C-terminal Arg residue was considered not so critical for the peptide's hypocholesterolemic activity.

Also green tea catechins and calcium from dairy products have been mentioned as cholesterol uptake inhibitors. Epigallocatechin gallate (EGCG) is the major catechin found in green tea. In humans, tea catechins have been shown to reduce body weight, visceral fat mass, and plasma cholesterol, insulin, and glucose levels. Additionally, several animal studies demonstrated that catechins inhibited cholesterol absorption and lowered plasma cholesterol levels. The current explanation is that epicatechins increase the fecal excretion of cholesterol and total lipids. In a very recent human, randomized crossover study (Am J Clin Nutr 2007; 85:678-687), it became clear that increased calcium intake from dairy products attenuates postprandial lipidemia, probably because of reduced fat absorbtion. Presumably the calcium acts by forming insoluble fatty acid soaps or by binding to the bile acids, hereby impairing micel formation. Calcium supplements in the form of calcium carbonate were found to be non effective suggesting that another anion as, for example, phosphate is required.

### SUMMARY OF THE INVENTION

The present invention relates to the novel peptides which have a length of between 4 and 8 amino acids and comprises the amino acid sequence -Xaa-Xbb-Xcc-Xdd-, whereby Xaa and Xdd can be His (H), Arg (R) or Lys (K), and Xbb and Xcc can be Pro (P) or Gly (G). Preferably the novel peptides have a length of between 4 and 6 amino acids, more peferably 4 or 5 amino acids. Preferably this peptide has a positive charge. In a prefered embodiment Xbb or Xdd is Pro (P), preferably Xbb and Xdd are Pro (P). In another prefered embodiment Xaa and/or Xdd is Arg (R). Prefered peptides are R-P-P-R-S, R-P-P-R, K-P-P-K, K-P-G-R, K-G-P-R, R-G-P-R and R-G-G-R.

The present invention also relates to a protein hydrolysate comprising the novel peptides which have a length of between 4 and 8 amino acids and comprising the amino acid sequence -Xaa-Xbb-Xcc-Xdd-. Preferably the protein hydrolysate has a DH of 5 to 50%, more preferably 10 to 40% and most preferably a DH of 20 to 35%. Preferably the protein hydrolysate or peptide mixture has an enhanced proline content. A preferred protein hydrolysate or peptide mixture has a molar proline content (on basis of total amino acid residues present) of more than 10%, more preferred protein hydrolysate or peptide mixture has a molar proline content of more than 20%, even more preferred protein hydrolysate or peptide mixture has a molar proline content of more than 30%. Hydroxyproline residues, as present in collagen and gelatin, are excluded from these proline contents. Furthermore the present invention relates to a peptide mixture comprising the novel peptides which have a length of between 4 and 8 amino acids and comprising the amino acid sequence -Xaa-Xbb-Xcc-Xdd-. Preferably the peptide mixture or hydrolysate of the invention comprises at least 1 mg/g protein of the peptide of the invention, more preferably at least 4 mg/g, even more preferably at least 10 mg/g protein, still more preferably at least 25 mg/g protein and most preferably at least 50 mg/g protein. In general the peptide mixture or hydrolysate will have less than 300 mg/g protein of the peptide of the invention. In case of synthetically produced peptide or purified isolated peptide an amount of 1 g/g protein is possible.

This peptide mixture or hydrolysate of the invention preferably comprises at least 30 wt % (dry matter) peptides having a MW of less than 2000 Da, more preferably 35 to 90% wt (dry matter) of the peptide mixture are peptides having a MW of less than 1500 Da.

The present invention also relates to a enzymatic process to produce the novel peptides which have a length of between 4 and 8 amino acids and comprising the amino acid sequence -Xaa-Xbb-Xcc-Xdd- preferably by enzymatic hydrolysis of a protein source. Furthermore the present invention relates to the novel peptides that are produced using chemical, chemo-enzymatical or peptide fermentation techniques.

The present invention further comprises a method of treatment or preventing of hypercholesterolemia, artherosclerosis, coronary heart disease or increasing plasma HDL (High Density Lipoprotein) which comprisises the intake of a peptide which have a length of between 4 and 8 amino acids and comprising the amino acid sequence -Xaa-Xbb-Xcc-Xdd- a protein hydrolysate which comprises this peptide, or a peptide mixture comprising this peptide or the intake of a nutraceutical, food or feed product which comprises said peptide, said protein hydrolysate or said peptide mixture.

Furthermore the the present invention provides a method for the preparation of a nutraceutical, dietary supplement, food or feed product for the treatment or preventing of hypercholesterolemia, artherosclerosis, coronary heart disease or increasing plasma HDL (High Density Lipoprotein) whereby a peptide which has a length of between 4 and 8 amino acids and comprising the amino acid sequence -Xaa-Xbb-Xcc-Xdd-, a protein hydrolysate which comprises said peptide, or a peptide mixture comprising said peptide is introduced into the nutraceutical, food or feed product.

### DETAILED DESCRIPTION OF THE INVENTION

Numerous clinical and epidemiologic studies have shown repeatedly that an elevated blood cholesterol level is one of the major risk factors associated with the development of artherosclerosis and coronary heart disease. Amongst others, these studies have identified low levels of high-density lipoprotein (HDL) as a risk factor. Conversely, high HDL cholesterol levels, i.e. levels of more than 60 mg HDL per dL (more than 1.55 mmol/L) are generally recognized as a positive factor. For example, the epidemiological studies reported in “High-density lipoprotein cholesterol and cardiovascular disease. Four prospective American studies” (Gordon et al., Ciculation 1089; 79; 8-15) showed that a 1 mg/dl (0.026 mmol/l) increment in HDL cholesterol was associated with a significant coronary heart disease risk decrement of 2-3.7% in men and 3-4.7% in women. Taking the 60 mg HDL per dL as an average, these data illustrate that a 1.7% HDL increase is already highly relevant for minimizing the risk of coronary heart disease.

As shown in the present application the novel peptides which have a length of between 4 and 8 amino acids and comprising the amino acid sequence -Xaa-Xbb-Xcc-Xdd- are quite effective in lowering the cholesterol uptake and increasing HDL levels. It is found that the novel peptides, which have a length of between 4 and 8 amino acids and comprising the amino acid sequence -Xaa-Xbb-Xcc-Xdd-, resist gastrointestinal proteolytic degradation and are thus expected to be stable in the human intestinal tract. The novel peptides which have a length of between 4 and 8 amino acids and comprising the amino acid sequence -Xaa-Xbb-Xcc-Xdd- are therefore very suitable for an effective lowering of the cholesterol uptake as well as increase of HDL levels and can for example be used in functional food, feed, in dietary supplement, as a nutraceutical or as a medicament.

We noted that when the peptides of the invention were used in combination with plant sterols or green tea extract or epigallocatechin gallate, the cholesterol uptake lowering and the HDL increasing effects were even enhanced.

Although the present invention is not restricted to any theory or explanation, a possible explanation of the effect is that the peptide which is between 4 and 8 amino acids long and comprise the amino acid sequence -Xaa-Xbb-Xcc-Xdd- is capable of preventing cholesterol uptake in the gastrointestinal tract.

The peptide which is between 4 and 8 amino acids long and comprise the amino acid sequence -Xaa-Xbb-Xcc-Xdd- is found to be stable in the gastroinstestinal tract which makes this peptide very suitable for having its cholesterol uptake decreasing effect all along the duodenum, the jejunum and the ileum. The peptide has preferably at least two positive charges due to the presence of least two amino acids selected from the group consisting of Arg (R) residue, Lys (K) residue or His (H) residue in its sequence. The remaining amino acids are preferably Pro (P) or Gly (G). The present text provides illustrative examples of possible combinations of the group of positively charged amino acids Arg, Lys and His on the one hand and the Pro and Gly residues known to confer protease resistancy to a peptide on the other hand. The functional similarities within these two groups of amino acids make the inventors believe that also the other combinations will give the beneficial effects according to the present invention. This supposition is confirmed by data as provided in the copending application WO 2007/107587.

The Codex alimentarius refers to various product categories such as “Foods for Special Nutritional Uses” and “Dietary Supplements”. Examples of Foods for Special

Nutritional Uses include the categories of sport foods, slimming foods, infant formula, food for diabetics and clinical foods. The term Dietary Supplement as used herein denotes a product taken by mouth that contains a compound or mixture of compounds intended to supplement the diet. The compound or mixture of compounds in these products may include: vitamins, minerals, herbs or other botanicals and amino acids. Dietary supplements can also be extracts or concentrates, and may be found in many forms such as tablets, capsules, softgels, gelcaps, liquids, or powders. Apart from these categories specified in the Codex Alimentarius, we also recognize “Functional Foods” and “Nutraceuticals”. In the present text the term Functional Food refers to foodstuffs that contain a functional compound that helps supporting a good health. The term Nutraceutical is used herein to denote the usefulness in both the nutritional and pharmaceutical field of application. The nutraceutical compositions according to the present invention may be in any form that is suitable for administrating to the animal body including the human body, especially in any form that is conventional for oral administration, e.g. in solid form such as (additives/supplements for) food or feed, food or feed premix, tablets, pills, granules, dragees, capsules, and effervescent formulations such as powders and tablets, or in liquid form such as solutions, emulsions or suspensions as e.g. beverages, pastes and oily suspensions. Controled (delayed) release formulations incorporating the hydrolysates according to the invention also form part of the invention. Furthermore, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

Thus, novel nutraceutical compositions comprising the novel peptides which have a length of between 4 and 8 amino acids and comprising the amino acid sequence -Xaa-Xbb-Xcc-Xdd- can find use as supplement to food and beverages and as pharmaceutical formulations or medicaments for enteral or parenteral application which may be solid formulations such as capsules or tablets, or liquid formulations, such as solutions, suspensions or emulsions.

It is now found that the peptide according to the invention may be used as an ingredient in various food products. Such products may be pasteurised or even be sterilized to guarantee adequate shelf stabilities without dramatic losses of the relevant peptide activity. In food products such as condiments, various beverages and emulsions such as low fat spreads, the peptide remains sufficiently active to achieve an adequate cholesterol lowering effect. Therefore, the peptides according to the invention are advantegeously combined with a large variety of food products, including those products incorporating cholesterol lowering stanols in their fat phase.

In general the taste of the food product is not affected or altered by the presence of the peptide With the necessary precautions, the peptide survives the sterilisation or pasteurization treatment and maintains its activity during the whole shelf life of the product.

The invention thus relates to sterilized, pasteurized and shelf stable food products containing the peptide of the invention.

A food product having an extended or improved shelf life is understood as having a shelf life of at least one week up to a year or more, during which the organoleptical properties as well as the microbial safety of the product are guaranteed. Obviously the allowable shelf life strongly depends on the actual storage conditions of the food product. Many perishable food products have to be stored cool in order to maximize their shelf lifes. Preferred food products are various types of dairy products. Dairy products are typically stored cool (preferably between 0 and 10 degrees Celcius) and comprise an aqueous as well as an fat phase hereby allowing the combination of the peptides according to the invention with hydrophobic stanols. Moreover, the peptides according to the invention are easily combined with dairy derived calcium to create synergy. Other preferred food products are condiments containing the peptide according to the invention. Condiments are food products used to enhance the flavour of other foods. Condiments have the advantage of being abundantly present at home and in restaurants, diners and supermarkets, and typically have of a prolonged shelf life. Yet another category of preferred food products are different types of beverages. Examples of beverages which are perfectly suitable as carriers for the present peptides are fruit juices and fruit concentrates. In fact even acidified or carbonated bottled water would present an excellent carrier for the peptides. “Shots” like vegetable or fruit concentrates also fall within this category. Likewise acid products containing a food grade preservative like benzoate or sorbate present excellent carriers for the peptides. Also very acid products that require no pasteurisation at all, such as cola's, present excellent carriers.

Food products that are especially preferred as a carrier for the peptide of the invention are those food products that allow a combination of the peptide according to the invention with green tea extracts such as epigallocatechin gallate or with calcium salts such as calcium phosphate or with plant sterols or stanols or esterified stanols. Such preferred products include oil-in-water (O/W) or water-in-oil (W/O) emulsions. Typical examples of such emulsions are spreads, preferably a margarine or a low fat spread. The peptide of the invention can also be consumed in the form of a beverage, a powder, a pil or a tablet to give the desired cholesterol lowering effect.

The novel peptides which have a length of between 4 and 8 amino acids and comprising the amino acid sequence -Xaa-Xbb-Xcc-Xdd- can be produced by various methods including economically viable production routes. Relevant options to produce the peptides according to the invention are the hydrolysis of suitable proteins using selected enzymes. Preferably the peptide fraction is further purified from the hydrolysate by advantageously using the characteristics of the desired peptide. Known methods such as ultrafiltration, chromatography, precipitation or extraction procedures rely on molecular size, charge and solubilities. Yield of the desired peptide fractions can also be enhanced by integrating multimers of nucleotide sequences encoding the desired peptide into the genome of the organism producing the relevant substrate protein. An example of this approach was recently published for the hypocholesterolemic Ile-Ala-Glu-Lys peptide from beta-lactoglobulin (Prak et al., 2007, Peptides 27, 1179-1186). Another option is presented by the chemical synthesis of the desired peptides. Production via chemical synthesis is possible using conventional techniques as for instance described in “Peptides: Chemistry and Biology” by N. Sewald and H. D. Jakubke, Eds. Wiley-VCH Verlag GmbH, 2002, Chapter 4. Particular cost-effective methods of chemical peptide synthesis suitable for large-scale production are based on the use of alkylchloroformates or pivaloyl chloride for the activation of the carboxylic group combined with the use of methyl esters for C-terminal protection and benzyloxycarbonyl (Z) or tert-butyloxycarbonyl groups for N-protection. Furthermore chemo-enzymatic peptide synthesis presents an interesting option because of the favorable cost structure that can be obtained by this approach. Also peptide fermentation and non-ribosomal peptide synthesis present interesting options in this context. The state of the art process for the production of peptides is by means of fermentation, wherein multimeric genes encoding tandem repeats of the peptide are fused to a polynucleotide encoding a carrier protein. This fusion protein is (over)produced by fermentation in a host cell, e.g. E. coli, after which the fusion protein is isolated and the peptides are cleaved from the carrier protein (Metlitskaia et al., Biotechnol. Appl. Biochem. 2004, 39:339-345).

Therefore, the novel peptides which have a length of between 4 and 8 amino acids and comprising the amino acid sequence -Xaa-Xbb-Xcc-Xdd- may be made by enzymatic hydrolysis or by fermentative approaches using any protein substrate containing the amino acid sequences -Xaa-Xbb-Xcc-Xdd-. Advantageously the protein substrate contains the fragments -Xaa-Xbb-Xcc-Xdd-. Prefered protein substrates for such enzymatic or fermentative approaches are formed by the proline-rich protein from cotton (K-P-P-K), tropoelastin from chicken (K-P-P-K), the 7 S storage protein from soy (K-P-G-R) and rice protein (R-G-G-R). Especially collagen (gelatin) from pig, chicken and pig form a good substrate as K-P-G-R, K-G-P-R and R-G-P-R can be obtained from this substrate. To release peptides according to the invention from these substrates, the substrate is conveniently incubated with trypsin (EC3.4.21.4). Trypsin is known to selectively cleave proteins at the C-terminal side of arginine (“R”) or lysine (“K”) residues. Precise cleavage at the N-terminal side of the peptides according to the invention is substrate dependent, i.e. depends on the amino acid sequence preceding the peptide. However, using subtilisin (EC3.4.21.62) for this purpose would provide a good option as this protease has a very broad selectivity which implies the generation of many, relatively short peptides. Important to note is that in this approach the peptides according to the invention will not be cleaved. The reason for this is that peptide bonds with proline (‘P”) or glycine (“G”) residues are known to be resistant to commonly used proteases such as trypsin or subtilisin. Both trypsin and subtilisin have slightly alkaline pH optima so that incubation of the substrate with the two enzymes can be carried out simultaneously. Enrichment of the desired peptide from the resulting hydrolysate can be achieved by a number of techniques. Peptides incorporating arginine or lysine residues can be separated from peptides that do not have such basic residues. For example, by adjusting the pH of the hydrolysate to a value between 4 and 6, more preferably between 5.0 and 5.5, peptides without such a basic residue will have no charge and, therefore, a reduced hydrophilicity. This feature can be used to our advantage, for example in a chromatographic or another separation process to isolate a large proportion of the arginine or lysine containing peptides. Charged arginine or lysine incorporating peptides can be removed by known techniques such as ion chromatography, hydrophobic interaction chromatography or electrodialysis. A practical background on the use of such characteristics in the chromatographic separation of the relevant peptides, can be found in a.o. the Protein Purification Handbook (issued by Amersham Pharmacia Biotech, nowadays GE Healthcare Bio-Sciences, Diegem, Belgium).

Because of the inherent resistancy of the peptides according to the invention against proteolytic attack, the desired peptides also can be obtained by approaches in which the less relevant parts of the protein substrate are removed. This can be accomplished by using complex proteolytic mixtures such as Sumizyme FP (Shin Nihon, Japan) or Flavourzyme (Novozymes, Denmark) that will truncate larger peptides to yield the proteolytically resistant peptides according to the invention. Alternatively the relevant protein substrates can be subjected to a fermentation with various types of highly proteolytic lactobacilli or streptococci. Through optimisation of the fermentation or hydrolysis conditions, the production of the biologically active peptides which have a length of between 4 and 8 amino acids and comprising the amino acid sequence -Xaa-Xbb-Xcc-Xdd- may be maximised. The skilled person trying to maximise the production will know how to adjust the process parameters, such as hydrolysis/fermentation time, hydrolysis/fermentation temperature, enzyme/microorganism type and concentration etc. The peptides which have a length of between 4 and 8 amino acids and comprising the amino acid sequence -Xaa-Xbb-Xcc-Xdd- or compositions comprising these peptides are advantageously hydrolysates and preferably made according to a process involving the following steps:

(a) enzymatic hydrolysis of a suitable protein substrate comprising -Xaa-Xbb-Xcc-Xdd- in its amino acid sequence resulting in a hydrolysed protein product comprising the peptides which have a length of between 4 and 8 amino acids and comprising the amino acid sequence -Xaa-Xbb-Xcc-Xdd-;

(b)—separation from the hydrolysed protein product of a fraction rich in peptides which have a length of between 4 and 8 amino acids and comprising the amino acid sequence -Xaa-Xbb-Xcc-Xdd-; and optionally

(c) concentrating and/or drying the hydrolysed protein product or a fraction rich in the desired peptides to obtain a concentrated liquid or a solid rich in peptides which have a length of between 4 and 8 amino acids and comprising the amino acid sequence -Xaa-Xbb-Xcc-Xdd-.

The enzymatic hydrolysis step may be any enzymatic treatment of the suitable protein substrate leading to hydrolysis of the protein resulting in liberation of peptides which have a length of between 4 and 8 amino acids and comprising the amino acid sequence -Xaa-Xbb-Xcc-Xdd-. As mentioned above several enzyme combinations can be used to release the desired peptides from the protein substrate. A suitable protein substrate may be any substrate encompassing the amino acid sequence -Xaa-Xbb-Xcc-Xdd-. Protein substrates known to encompass the peptides according to the invention can be obtained from, for example, cotton protein, rice protein, mouse ear cress protein, tropoelastin, the proline-rich protein from cotton, the 7 s storage protein from soy as well as collagen preparations obtained from cow, pig or chicken. Instead of collagen, preferably gelatin derived from the relevant source, for example cow, pig or chicken collagen, is used. The enzyme may be any enzyme or enzyme combination that is able to hydrolyse protein resulting in the liberation of one or more of the peptides of the invention.

The separation step (b) may be executed in any way known to the skilled person, e.g. by precipitation, filtration, centrifugation, extraction or chromatography and combinations thereof. Preferably the separation step (b) is executed using micro- or ultrafiltration techniques. The pore size of the membranes used in the filtration step, as well as the charge of the membrane may be used to control the separation of the peptides of the invention. Another preferred separation step is cation chromatography in which use is made of the positive charge of the peptide according to the invention under acid pH conditions. The concentration step (c) may involve nanofiltration or evaporation of the fraction generated by step (b) to yield a highly concentrated liquid.

If suitably formulated, e.g. with a low water activity (Aw), a low pH and preferably a preservative such as benzoate or sorbate, such concentrated liquid compositions form an attractive way of storage of the peptides according to the invention. Optionally the evaporation step is followed by a drying step e.g. by spray drying or freeze drying to yield a solid containing a high concentration of the peptides of the invention.

The enzymatic hydrolysis process comprises preferably a single enzyme incubation step. The process according to the invention has in general an incubation time of less than 24 hours, preferably the incubation time is less than 10 hours and more preferably less than 4 hours. The incubation temperature is in general higher than 30° C., preferably higher than 40° C. and more preferably higher than 50° C.

By protein hydrolysate, hydrolysate or hydrolysed protein is meant the product that is formed by enzymatic hydrolysis of the protein, an enriched hydrolysate being a fraction of the protein hydrolysate for example enriched in selected peptides or wherein peptides or polypeptides have been removed from the hydrolysate. So an enriched hydrolysate is preferably a mixture of peptides (or a peptide mixture). The peptide mixture of the invention is therefore a mixture of at least two peptides whereof at least one is a peptide comprising the amino acid sequence -Xaa-Xbb-Xcc-Xdd-.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. The one-letter code of amino acids used herein is commonly known in the art and can be found in Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989). An endoprotease is defined herein as an enzyme that hydrolyses peptide bonds in a polypeptide in an endo-fasion and belongs to the group EC 3.4. The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25). Exoproteases are defined herein as enzymes that hydrolyze peptide bonds adjacent to a terminal a-amino group (“aminopeptidases”), or a peptide bond between the terminal carboxyl group and the penultimate amino acid (“carboxypeptidases”). To prepare the hydrolysates incorporating the peptides according to the present invention the endoproteases trypsin (EC 3.4.21.4) and subtilisin (EC 3.4.21.62) are preferred. Truncation of the peptides thus produced can be achieved with a suitable amino- and/or carboxypeptidase.

According to the present process preferably at least 20%, more preferably at least 30%, most preferably at least 40% of an -Xaa-Xbb-Xcc-Xdd- containing protein sequence is converted into peptides which have a length of between 4 and 8 amino acids and comprising the amino acid sequence -Xaa-Xbb-Xcc-Xdd-.

After elution from the chromatographic resin, decantation, filtration or low speed centrifugation, the liquids containing the biologically active peptides, an enriched hydrolysate can be recovered in a purified state. A subsequent evaporation and spray drying step will yield an economical route for obtaining a food grade concentrate, paste or powder with a high bio-activity. The proper formulation of such a concentrate for example by lowering the water activity (Aw) in combination with a pH adjustment or the addition of a food grade preservative like a benzoate or a sorbate will yield a microbiologically stabilized, food grade, liquid concentrate of the peptides of the invention. If required, the supernatant obtained after the decantation, filtration or low speed centrifugation can be further processed to improve the palatability of the final product. For example, the supernatant can be contacted with powdered activated charcoal followed by a filtration step to remove the charcoal. If required, the concentration of the bioactive ingredients can be increased even further by subsequent purification steps in which use is made of the specific amino acid composition of the peptides of the invention. We have found that peptides which have a length of between 4 and 8 amino acids and comprising the amino acid sequence -Xaa-Xbb-Xcc-Xdd- survive incubation conditions simulating the digestive conditions typically found in the gastro-intestinal tract. On the basis of these data we conclude that the novel peptides are likely to survive in the mammalian (for example human) gastrointestinal tract implying a considerable economic potential if used to treat the health risks typically associated with increased plasma cholesterol levels or low HDL cholesterol levels.

The peptides which have a length of between 4 and 8 amino acids and comprising the amino acid sequence -Xaa-Xbb-Xcc-Xdd- as obtained either before or after an additional (for example chromatographic) purification step may be used for the incorporation into food products that are widely consumed on a regular basis. Examples of such products are margarines, spreads, various dairy products such as butter or yoghurts or milk or whey containing beverages. Furthermore the peptides may be incorporated in mineral waters. Although such compositions are typically administered to human beings, they may also be administered to animals, preferably mammals, to counteract high cholesterol levels. Furthermore the high concentration of peptides suitable for lowering cholesterol uptake, makes these products very useful for the incorporation into dietary supplements in the form off pills, tablets or highly concentrated solutions or pastes or powders. Slow release dietary supplements that will ensure a continuous release of these peptides are of particular interest. The peptides which have a length of between 4 and 8 amino acids and comprising the amino acid sequence -Xaa-Xbb-Xcc-Xdd- according to the invention may be formulated as a dry powder in, for example, a pill, a tablet, a granule, a sachet or a capsule. Alternatively the peptides according to the invention may be formulated as a liquid in, for example, a syrup or a capsule. The compositions used in the various formulations and containing the peptides according to the invention may also incorporate at least one compound of the group consisting of a physiologically acceptable carrier, adjuvant, excipient, stabiliser, buffer and diluant which terms are used in their ordinary sense to indicate substances that assist in the packaging, delivery, absorption, stabilisation, or, in the case of an adjuvant, enhancing the physiological effect of the peptides, such as catechins or calcium salts. The relevant background on the various compounds that can be used in combination with the peptides according to the invention in a powdered form can be found in “Pharmaceutical Dosage Forms”, second edition, Volumes 1, 2 and 3, ISBN 0-8247-8044-2 Marcel Dekker, Inc.

Preferably, the nutraceutical or food product comprises from 2 to 30 wt % plant-derived sterol, more preferably from 4 to 20 wt % plant-derived sterol. Incorporation of plant-derived sterol maximizes reductions in LDL-cholesterol achieveable by dietary means. Sterols represent a group of compounds that are alcoholic derivatives of cyclopentanoperhydrophenanthrene and are an essential constituent of cell membranes in animals and plants (A. H. Lichtenstein, Circulation, 2000, 103, p. 1117). Cholesterol is the sterol of mammalian cells, whereas multiple sterols, or phytosterols, are produced by plants, with sitosterol, campesterol, and stigmasterol being most common. Plant sterols, although structurally similar to cholesterol, are not synthesized by the human body. They are very poorly absorbed by the human intestine. The specific plant sterols that are currently incorporated into foods intended to lower blood cholesterol levels are extracted from soybean oil or tall (pine tree) oil. Additional sources of plant sterols may be available in the near future. The plant sterols currently incorporated into foods are esterified to unsaturated fatty acids (creating sterol esters) to increase lipid solubility, thus allowing maximal incorporation into a limited amount of fat. Some plant sterols currently available are saturated, to form the stenol derivatives, sitostanol and campestanol, which after esterification form stanol esters. These stanols interfere with small intestine absorption of intestinal and biliary cholesterol. While they lower LDL (Low Density Lipoprotein) levels, they have no significant effect on HDL (High Density Lipoprotein) or triglyceride levels (N Eng J Med 1995; 333: 1308-1312). Quite surprisingly our data (see Examples 5 and 6) demonstrate that the peptides according to the invention have the capacity to significantly increase HDL plasma levels. Diabetes patients as well as physically inactive individuals persons and smokers are known to suffer from low HDL levels. Using the peptides according to the present invention compensates for such low HDL levels. Also some drugs like beta blockers, thiazide, diuretica and anabol steroids may result in lowered plasma HDL level. For all these persons the present invention offers a solution to increase the plasma HDL level. We herewith want to make a note that of course we do not want encourage unhealthy life styles like smoking or remaining physically inactive.

The above described nutraceutical ingredients contributing to increasing cardiovascular health as well as K+, Ca2+ and Mg2+, B-vitamins (folic acid, B6, B12) and sterols/stanols are herein collectively referred to as heart health ingredients.

### Materials and Methods

Materials

All synthetic peptides used in the following Examples were obtained from Pepscan Presto B. V., Lelystad, The Netherlands.

Methods

LC/MS/MS analysis used in the detection of peptides with a length of between 4 and 8 amino acids and comprises the amino acid sequence -Xaa-Xbb-Xcc-Xdd.

HPLC using an ion trap mass spectrometer (Thermoquest®, Breda, the Netherlands) coupled to a P4000 pump (Thermoquest®, Breda, the Netherlands) was used in quantification of the peptides of interest in the enzymatic protein hydrolysates produced. The peptides formed were separated using a lnertsil 3 ODS 3, 3 μm, 150*2.1 mm (Varian Belgium, Belgium) column in combination with a gradient of 0.1% formic acid in Milli Q water (Millipore, Bedford, Mass., USA; Solution A) and 0.1% formic acid in acetonitrile (Solution B) for elution. The gradient started at 100% of Solution A, kept here for 5 minutes, increasing linear to 5% B in 10 minutes, followed by linear increasing to 45% of solution B in 30 minutes and immediately going to the beginning conditions, and kept here 15 minutes for stabilization. The injection volume used was 50 microliters, the flow rate was 200 microliter per minute and the column temperature was maintained at 55° C. The protein concentration of the injected sample was approx. 50 micrograms/milliliter.

Detailed information on the individual peptides was obtained by using dedicated MS/MS for the peptides of interest, using optimal collision energy of about 30%. Quantification of the individual peptides was performed using external calibration, by using the most abundant fragment ions observed in MS/MS mode.

Prior to LC/MS/MS the enzymatic protein hydrolysates were centrifuged at ambient temperature and 13000 rpm for 10 minutes, filtered through a 0.22 μm filter and the supernatant was diluted 1:100 with MilliQ water.

Kjeldahl Nitrogen

Total Kjeldahl Nitrogen was measured by Flow Injection Analysis. Using a Tecator FIASTAR 5000 Flow Injection System equipped with a TKN Method Cassette 5000-040, a Pentium 4 computer with SOFIA software and a Tecator 5027 Autosampler the ammonia released from protein containing solutions was quantitated at 590 nm. A sample amount corresponding with the dynamic range of the method (0.5-20 mg N/I) is placed in the digestion tube together with 95-97% sulphuric acid and a Kjeltab subjected to a digestion program of 30 minutes at 200 degrees C. followed by 90 minutes at 360 degrees C. After injection in the FIASTAR 5000 system the nitrogen peak is measured from which the amount of protein measured can be inferred.

Amino Acid Analysis

A precisely weighed sample of the proteinaceous material was dissolved in dilute acid and precipitates were removed by centrifugation in an Eppendorf centrifuge. Amino acid analysis was carried out on the clear supernatant according to the PicoTag method as specified in the operators manual of the Amino Acid Analysis System of Waters (Milford Mass., USA). To that end a suitable sample was obtained from the liquid, then dried and subjected to vapour phase acid hydrolysis and derivatised using phenylisothiocyanate. The various derivatised amino acids present were quantitated using HPLC methods and added up to calculate the total level of free amino acids in the weighed sample. The amino acids Cys and Trp are not included in the data obtained in this analysis.

The Degree of Hydrolysis (DH) of the protein hydrolysate was measured using a rapid OPA test and calculated as described (Nielsen et al, JFS, Vol 66, NO 5, 642-646, 2001).

Molecular weight distribution of peptides and proteins present in hydrolysates. Analysis of the peptide size distribution of protease treated protein samples was done on an automated HPLC system equipped with a high pressure pump, injection device able to inject 10-100 μl sample and a UV detector able to monitor the column effluent at 214 nm. The column used for this analysis was a Superdex Peptide HR 10/300 GL (Amersham) equilibrated with 20 mM Sodium Phosphate/250 mM Sodium Chloride pH 7.0 buffer. After injecting a sample (typically 50 μl) the various components were eluted from the column with buffer in 90 min at a flow rate of 0.5 ml/min. The system was calibrated using a mixture of cytochrome C (Mw 13 500 Da), aprotinin (Mw 6510 Da) and tetra-glycine (Mw 246 Da) as molecular weight markers.

The following Examples illustrate the invention further.

### Examples

### Example 1

### Soft Gelatin Capsule

Soft gelatin capsules are prepared by conventional procedures using ingredients specified below:

Active ingredients: a peptide which is between 4 and 8 amino acids long and comprise the amino acid sequence -Xaa-Xbb-Xcc-Xdd- 0.1 g, protein hydrolysates 0.3 g

Other ingredients: glycerol, water, gelatin, vegetable oil.

### Example 2

### Hard Gelatin Capsule

Hard gelatin capsules are prepared by conventional procedures using ingredients specified below:

Active ingredients: a peptide which is between 4 and 8 amino acids long and comprise the amino acid sequence -Xaa-Xbb-Xcc-Xdd- 0.3 g, protein hydrolysates 0.7 g

Fillers: lactose or cellulose or cellulose derivatives q.s

Lubricant: magnesium stearate if necessary (0.5%)

### Example 3

### Tablet

Tablets are prepared by conventional procedures using ingredients specified below:

Active ingredients: a peptide which is between 4 and 8 amino acids long and comprise the amino acid sequence -Xaa-Xbb-Xcc-Xdd- 0.4 g, unhydrolysed protein 0.4 g

Other ingredients: microcrystalline cellulose, silicone dioxide (SiO2), magnesium stearate, crosscarmellose sodium.

### Example 4

### Soft Drink with 30% Juice

Typical serving: 240 ml

Active ingredients:

a peptide which is between 4 and 8 amino acids long and comprise the amino acid sequence -Xaa-Xbb-Xcc-Xdd- and protein hydrolysates and maltodextrin as a carbohydrate source are incorporated in this food item:

A peptide which is between 4 and 8 amino acids long and comprise the amino acid sequence -Xaa-Xbb-Xcc-Xdd-: 0.5-5 g/per serving

Protein hydrolysates: 1.5-15 g/per serving

Maltodextrin: 3-30 g/per serving

1.0 A Soft Drink Compound is prepared from the following ingredients:

Juice concentrates and water soluble flavors:

| | [g] |
|---|---|
| 1.1 Orange concentrate | |
| 60.3 Brix, 5.15% acidity | 657.99 |
| Lemon concentrate | |
| 43.5 Brix, 32.7% acidity | 95.96 |
| Orange flavor, water soluble | 13.43 |
| Apricot flavor, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavors | |
| Orange flavor, oil soluble | 0.34 |
| Orange oil distilled | 0.34 |

1.6 Active Ingredients

Active ingredients (this means the active ingredient mentioned above: a peptide which is between 4 and 8 amino acids long and comprise the amino acid sequence -Xaa-Xbb-Xcc-Xdd-) and protein hydrolysates and maltodextrin in the concentrations mentioned above.

Fruit juice concentrates and water soluble flavors are mixed without incorporation of air. The color is dissolved in deionized water. Ascorbic acid and citric acid is dissolved in water. Sodium benzoate is dissolved in water. The pectin is added under stirring and dissolved while boiling. The solution is cooled down. Orange oil and oil soluble flavors are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenizing using a Turrax and then a high-pressure homogenizer (p₁=200 bar, p₂=50 bar).

2.0 A Bottling Syrup is Prepared from the Following Ingredients:

| | [g] |
|---|---|
| Softdrink compound | 74.50 |
| Water | 50.00 |
| Sugar syrup 60 Brix | 150.00 |

The ingredients of the bottling syrup are mixed together. The bottling syrup is diluted with water to 1 l of ready to drink beverage.

Variations:

Instead of using sodium benzoate, the beverage may be pasteurized. The beverage may also be carbonized.

### Example 5

### Hypocholesterolemic Effects of Peptide R-P-P-R-S in Fisher F344 Rats

In this experiment the effects of the chemically synthesized tripeptide R-P-P-R-S was tested on plasma cholesterol levels in the hypercholesterolemic rat model.

In short male Fisher rats (F344) from Charles River (Sulzfeld, Germany), weighing 65-96 g were housed individually with free access to water and feed, with an alternating 12-hour light-dark cycle. The animals were fed a rodent atherogenic diet high in cholesterol and fat for the 2 weeks preceding the treatment. The macro-nutrient composition of the diet was as follows (g/100 g anhydrous mix): protein 20.8; fiber 4.2; fat 15; carbohydrate 48. The diet also contained 1.25 wt % cholesterol, 0.5 wt % sodium cholate and a standard vitamin and mineral mix, prepared according to rat nutritional requirements. The main fats consisted of cacoa butter (7.5 wt %) and soja oil (4.35 wt %). After 2 weeks of adaptation to the diet the rats were randomized according to body weight into two experimental groups of 10 animals. The two groups were treated orally every morning for 7 days with either water (control group) or peptide R-P-P-R-S, at a dose of 100 mg/kg body weight (BW) while continuing the aetherogenic diet. There was no difference in feed consumption over the whole experiment. The day before the last administration, all animals were dosed orally in the evening and deprived of food overnight (14 hours) but allowed free access to water. At day 7 the fasted rats were treated again orally in the morning and after two hours the animals were sacrificed by withdrawing blood from the vena Cava under Isoflurane anesthesia. Blood was collected into tubes containing EDTA as an anticoagulant. Plasma was prepared from the blood by immediate centrifugation at 1600 g for 10 minutes at 4° C. Assays of plasma cholesterol and HDL-cholesterol (precipitation method) were determined enzymatically on a Hitachi 912 automatic analyzer (Roche Diagnostica, Switzerland). Non-HDL cholesterol was calculated by difference between total cholesterol and HDL cholesterol. One animal in the control group was excluded from the analysis due to hemolytic plasma which interfered with the measurements. All data are expressed as means±standard deviation (SD) for animals in each diet group, with n being the number of rats. Statistical significance of the mean differences between dietary groups was tested by one-way analysis of variance (ANOVA). If significant differences were found, the Dunnett's test for multiple comparison was used to compare each group to the control group. P values less than 0.05 were considered significant (Table 1).

The tested peptide (100 mg/Kg BW) significantly lowered plasma total cholesterol levels and non-HDL cholesterol (VLDL-cholesterol+LDL-cholesterol) levels as compared to the control group. Plasma HDL-cholesterol was significantly increased after treatment with the experimental peptides when compared to the control group. The data suggest that the peptide strongly reduce risk factors for cardiovascular problems and atherosclerosis. In a dose response experiment the effective dose range of the peptide can be determined.

TABLE 1
Effects of peptide R-P-P-R-S on plasma total cholesterol
and lipoproteins cholesterol levels in rats^{a}

| | Total cholesterol | non HDL cholesterol | HDL cholesterol |
|---|---|---|---|
| Control | 10.1 ± 2.2 | 10.0 ± 2.2 | 0.14 ± 0.08 |
| RPPRS | 2.9 ± 0.4^{b} | 2.5 ± 0.5^{b} | 0.36 ± 0.10^{b} |

^{a}Values are expressed as mean ± SD given in mmol/L (n = 9-10 per group)
^{b}significantly different from control (p < 0.05)

### Example 6

### Hypocholesterolemic Effects of Peptide R-P-P-R and K-P-P in Fisher F344 Rats

In this experiment the effects of the chemically synthesized peptides R-P-P-R and K-P-P, the free amino acid L-arginine and the potent and selective cholesterol absorption inhibitor Ezetimib (Ezetrol, MSD Germany) were tested on plasma cholesterol levels in the hypercholesterolemic rat model.

Male Fisher rats (F344) from Charles River (Sulzfeld, Germany), weighing 65-96 g were housed individually with free access to water and feed, with an alternating 12-hour light-dark cycle. The animals were fed a rodent atherogenic diet high in cholesterol and fat similar to the procedure described in Example 5. After 2 weeks of adaptation to the diet the rats were randomized according to body weight into five experimental groups of 10 animals each. The five groups were treated orally every morning for 8 days with either water (control group) or with R-P-P-R, K-P-P and L-arginine at a dose of 100 mg/kg body weight (BW) or with Ezetimib at a dose of 1 mg/kg BW while continuing the aetherogenic diet. There was no difference in feed consumption over the whole experiment. The day before the last administration, all animals were dosed orally in the evening and deprived of food for the last 6 hours but allowed free access to water. At day 8 the fasted rats were treated again orally in the morning and after two hours the animals were sacrificed by withdrawing blood from the vena Cava under Isoflurane anesthesia. Blood was collected into tubes containing EDTA as an anticoagulant. Plasma was prepared from the blood by immediate centrifugation at 1600 g for 10 minutes at 4° C. Assays of plasma cholesterol and HDL-cholesterol (precipitation method) were determined enzymatically on a Hitachi 912 automatic analyzer (Roche Diagnostica, Switzerland). Non-HDL cholesterol was calculated by difference between total cholesterol and HDL cholesterol. All data are expressed as means±standard deviation (SD) for animals in each diet group, with n being the number of rats.

R-P-P-R (100 mg/kg BW) significantly lowered plasma total cholesterol and non-HDL cholesterol (VLDL-cholesterol+LDL-cholesterol) levels as compared to the control group. Moreover, R-P-P-R tended to increase plasma HDL-cholesterol as compared to the control group. K-P-P (100 mg/kg BW) tended to lowered plasma total cholesterol and non-HDL cholesterol levels as compared to the control group while plasma HDL-cholesterol levels were significantly increased as compared to the control group. L-arginine had no effect on plasma total cholesterol, non-HDL cholesterol and HDL-cholesterol levels as compared to the control group. As expected Ezetimib (1 mg/kg BW) had prominent effects on total cholesterol, non-HDL cholesterol and HDL-cholesterol levels. As demonstrated in several human epidemiologic trials, HDL increases by only a few percent significantly lower the risk of coronary heart disease. Against this background, the data obtained here strongly suggest that the dietary intake of peptides R-P-P-R and K-P-P can strongly reduce risk factors for cardiovascular problems and atherosclerosis. In contrast with the peptides studied, the addition of free arginine (“R”) to the diet did not affect either the total cholesterol or the HDL cholesterol levels.

TABLE 2
Effects of R-P-P-R, KPP, L-arginine and Ezetimib on plasma total
cholesterol and lipoproteins cholesterol levels in rats^{a}

| | Total cholesterol | non HDL cholesterol | HDL cholesterol |
|---|---|---|---|
| Control | 7.41 ± 1.69 | 7.09 ± 1.68 | 0.31 ± 0.03 |
| RPPR | 5.28 ± 0.81 | 4.94 ± 0.81 | 0.35 ± 0.04 |
| KPP | 6.82 ± 1.42 | 6.45 ± 1.44 | 0.37 ± 0.04 |
| L-arginine | 7.66 ± 1.08 | 7.32 ± 1.08 | 0.33 ± 0.05 |
| Ezetimib | 4.70 ± 0.93 | 4.19 ± 0.90 | 0.51 ± 0.06 |

^{a}Values are expressed as mean ± SD given in mmol/L (n = 10 per group)

1. A peptide which has a length of between 4 and 8 amino acids, preferably between 4 and 6 amino acids and more preferably between 4 or 5 amino acids, and comprises the amino acid sequence -Xaa-Xbb-Xcc-Xdd-, whereby Xaa and Xdd can be His (H), Arg (R) or Lys (K), and Xbb and Xcc can be Pro (P) or Gly (G).

2. The peptide according to claim 1 which has a positive charge.

3. The peptide according to claim 1 whereby Xbb and/or Xcc is Pro (P).

4. The peptide according to claim 1 whereby Xaa and/or Xdd is Arg (R).

5. The peptide according to claim 1 which is R-P-P-R-S (SEQ ID NO: 1), R-P-P-R (SEQ ID NO: 2), K-P-P-K (SEQ ID NO: 3), K-P-G-R (SEQ ID NO: 4), K-G-P-R (SEQ ID NO: 5), R-G-P-R (SEQ ID NO: 6) and R-G-G-R (SEQ ID NO: 7).

6. The peptide according to claim 1 which is a pharmaceutically active compound.

7. A protein hydrolysate comprising a peptide according to claim 1.

8. A protein hydrolysate according to claim 7 which has a DH of 5 to 50%, preferably 10 to 40%, more preferably a DH of 20 to 35%.

9. A peptide mixture comprising at least one peptide according to claim 1.

10. A peptide mixture of claim 9 which comprises at least 1 mg of said peptide/gram protein, preferably at least 2 mg of said peptide/gram protein, more preferably at least 4 mg/g, even more preferably at least 10 mg/g protein, still more preferably at least 25 mg/g protein and most preferably at least 50 mg/g protein.

11. A peptide mixture according to claim 9 whereby the amount of peptides which have a MW of less than 2000 Da is at least 30 wt % (dry matter) of the peptide mixture, preferably between 35 and 70% wt (dry matter) of the peptide mixture.

12. A peptide mixture according to claim 9 which comprises 1 to 90% of water, preferably 1 to 30% of water, more preferably 1 to 15% of water.

13. A method to produce a peptide which has a length of between 4 and 8 amino acids, preferably between 4 and 6 amino acids and more preferably between 4 or 5 amino acids, and comprises the amino acid sequence -Xaa-Xbb-Xcc-Xdd-, whereby Xaa and Xdd can be His (H), Arg (R) or Lys (K), and Xbb and Xcc can be Pro (P) or Gly (G), which comprises the enzymatic hydrolysis of a suitable protein.

14. A method according to claim 12 which comprises the use of a protease to hydrolyze a suitable protein, preferably at least two endoproteases are used.

15. A method of treatment or prevention of hypercholesterolemia, artherosclerosis, coronary heart disease or increasing plasma HDL (High Density Lipoprotein) which comprises intake of a peptide according to claim 1, a protein hydrolysate comprising the peptide, or a peptide mixture comprising at least one of the peptide or intake of a nutraceutical, dietary supplement, food or feed product which comprises the peptide, the protein hydrolysate, or the peptide mixture.

16. A method for preparation of a nutraceutical, dietary supplement, food or feed product for treatment or prevention of hypercholesterolemia, artherosclerosis, coronary heart disease or increasing plasma HDL (High Density Lipoprotein) whereby a peptide according to claim 1, a protein hydrolysate comprising the peptide, or a peptide mixture comprising at least one of the peptide is introduced in said nutraceutical, dietary supplement, food or feed product.

17. A nutraceutical, dietary supplement, food or feed product for treatment or prevention of hypercholesterolemia, artherosclerosis, coronary heart disease or increasing plasma HDL (High Density Lipoprotein) comprising a peptide according to claim 1; a protein hydrolysate comprising the peptide, or a peptide mixture comprising at least one of the peptide.
